(19) United States
(12) Patent Application Publication
(10) Pub. No.: US 2026/0096867 A1
(43) Pub. Date: Apr. 9, 2026

(54) REMOVAL TOOL FOR MAGNETIC LOCALIZATION MARKER

(71) Applicant: Stryker Corporation, Portage, MI (US)

(72) Inventors: Ananth RAVI, Toronto (CA); John DILLON, Mississauga (CA); Michael GIANNINI, Toronto (CA); Brian CHAN, Toronto (CA)

(73) Assignee: Stryker Corporation, Portage, MI (US)

(21) Appl. No.: 19/126,628

(22) PCT Filed: Nov. 2, 2023
(86) PCT No.: PCT/IB2023/000675

§ 371 Date: May 1, 2025

Related U.S. Application Data
(60) Provisional application No. 63/382,071, filed on Nov. 2, 2022.

(51) Int. Cl. A61B 90/00

(52) U.S. Cl. CPC A61B 90/39; A61B 2090/3954; A61B 2090/3987

(57) ABSTRACT

Disclosed is a device and method for retrieving a magnetic marker implanted in an individual. The device includes an introducer having a tip. An obturator is disposed within the introducer. The obturator has a magnetic end. The obturator is operable such that the magnetic end is enclosed within the introducer in a first position and the magnetic end extends at least partially beyond the tip of the introducer in a second position. A method for removing a magnetic marker from a location within an individual includes inserting an introducer into an individual. The introducer has an obturator with a magnetic end. A magnetic marker is magnetically attracted using the obturator such that the magnetic marker attaches to the obturator. The obturator is retracted into the introducer such that the magnetic marker is at least partially within the introducer.

### CROSS-REFERENCE TO RELATED APPLICATIONS

This application claims priority to U.S. Provisional Application No. 63/382,071, filed on Nov. 2, 2022, now pending, the disclosure of which is incorporated herein by reference.

### FIELD OF THE DISCLOSURE

The present disclosure relates to radiological localization using a magnetic marker, and more particularly to the ability to remove a magnetized marker (e.g., a magnetic localization marker) non-surgically or with minimally invasive techniques.

### BACKGROUND OF THE DISCLOSURE

During localization procedures, such as breast localization procedures, there is a need to mark lesions with a localization marker in radiology for identification during surgery and later removal. To date, a magnetic marker has been used to achieve this goal and a customized system is then used to identify the position and remove the marker and associated lesion with accuracy (e.g., MOLLI Marker and System).

The accuracy of the surgical removal of the lesion is predicated on the implantation of the magnetic marker at its intended target. However, due to unforeseen events that may occur in radiology (e.g., communication gap in terms of appropriate target, challenging imaging, complex anatomy, etc.), this placement may be sub-optimal. There is a need for the ability to remove an implanted magnetic marker and/or correct the position of an implanted magnetic marker.

Current marker removal techniques require a surgical approach, which places an undue burden on patients. To date, no markers can be removed from the patient after implantation minimally-invasively.

### BRIEF SUMMARY OF THE DISCLOSURE

In an aspect, the present disclosure may be embodied as a device for retrieving a magnetic marker implanted in an individual. The device includes an introducer having a tip. An obturator is disposed within the introducer. The obturator has a magnetic end. The obturator is operable such that the magnetic end is enclosed within the introducer in a first position of the obturator and the magnetic end extends at least partially beyond the tip of the introducer in a second position of the obturator.

In some embodiments, the obturator has a shaft and a permanent magnet affixed to the shaft at the magnetic end of the obturator. For example, the permanent magnet may be adhered at an end of the shaft. The permanent magnet may be adhered at the shaft using an ultraviolet light-curable adhesive, a cyanoacrylate-based adhesive, an epoxy, an acrylic adhesive. The permanent magnet is adhered to the end of the shaft. In some embodiments, the end of the shaft is cup-shaped. A portion of the permanent magnet may be shaped to correspond with the cup-shaped end of the shaft.

In some embodiments, the shaft is hollow and the permanent magnet is at least partially disposed within an end of the hollow shaft. In some embodiments, a rod is disposed within the hollow shaft.

In some embodiments, the permanent magnet is mechanically affixed at an end of the shaft. For example, the permanent magnet may be mechanically affixed using screw threads, a dovetail fitting, an interference fit, or other technique or combinations thereof.

The permanent magnet may be made from or include neodymium. In some embodiments, the permanent magnet comprises a bio-compatible material. For example, the permanent magnet may include an enclosure (such as, for example, a coating) made from a bio-compatible material. The bio-compatible material may be, for example, surgical stainless steel, titanium, nitinol, polycarbonate, an acetyl-copolymer, PEEK, or combinations thereof.

In some embodiments, the device includes an ejector arranged through the obturator and operable to extend beyond the magnetic end of the obturator. For example, the ejector may be coaxial with the obturator. The ejector may be made from a bio-compatible material, such as, for example, surgical stainless steel, titanium, nitinol, polycarbonate, an acetyl-copolymer, PEEK, or combinations thereof. In some embodiments, the ejector is made from a non-magnetic material.

The shaft may be made from a bio-compatible material, such as, for example, surgical stainless steel, titanium, nitinol, polycarbonate, an acetyl-copolymer, PEEK, or combinations of thereof.

In some embodiments, the permanent magnet is made up of two or more magnets.

In another aspect, the present disclosure may be embodied as a method for removing a magnetic marker from a location (i.e., a first location) within an individual. The method includes inserting an introducer into an individual. The introducer has an obturator with a magnetic end. The introducer, obturator, and/or magnetic end may be of any configuration disclosed herein.

The method includes attracting, magnetically, the magnetic marker using the obturator such that the magnetic marker attaches to the obturator. The obturator is retracted into the introducer such that the magnetic marker is at least partially within the introducer. In some embodiments, the method includes repositioning the introducer to move the magnetic marker to a second position within the individual. An ejector may be extended (e.g., from the introducer, from the obturator, etc.) to detach the magnetic marker from the obturator (e.g., by pushing the magnetic marker away from the magnetic end of the obturator). In some embodiments, the method includes removing the introducer from the individual.

### DESCRIPTION OF THE DRAWINGS

For a fuller understanding of the nature and objects of the disclosure, reference should be made to the following detailed description taken in conjunction with the accompanying drawings, in which:

FIG. 1 depicts a device according to an embodiment of the present disclosure;

FIG. 2 depicts an obturator according to an embodiment of the present disclosure, wherein a permanent magnet abuts an end of a shaft;

FIG. 3 is a cross-sectional view of an obturator according to another embodiment of the present disclosure, wherein the shaft of the obturator has a cup-shaped end;

FIG. 4 is a cross-sectional view of an obturator according to another embodiment of the present disclosure, wherein the permanent magnet has a shape corresponding to a cupped end of the shaft;

FIG. 5 is a cross-sectional view of an obturator according to another embodiment of the present disclosure, wherein the shaft is hollow;

FIG. 6 is a cross-sectional view of an obturator according to another embodiment of the present disclosure, wherein a rod is disposed within a hollow shaft;

FIG. 7A is a cross-sectional view of an obturator according to another embodiment of the present disclosure, wherein the obturator including an ejector;

FIG. 7B is a cross-sectional view of the obturator of FIG. 7A, wherein the ejector is shown in a deployed position (i.e., advanced beyond the magnetic end of the obturator); and

FIG. 8 is a chart depicting a method according to another embodiment of the present disclosure.

### DETAILED DESCRIPTION OF THE DISCLOSURE

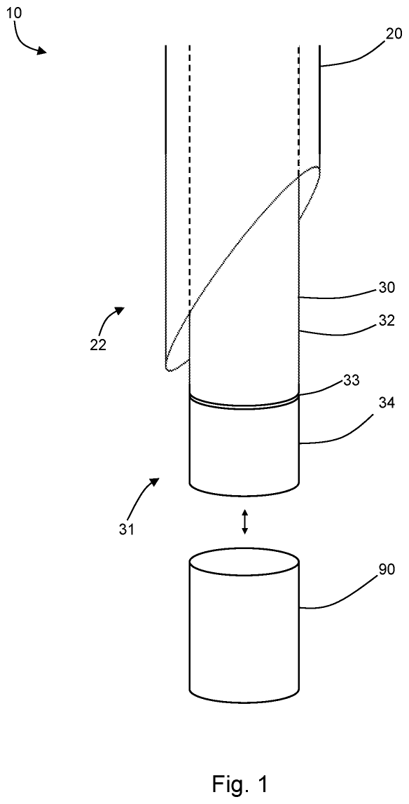

With reference to FIG. 1, the present disclosure may be embodied as a device 10 for removing a magnetic marker 90 implanted in an individual. The device 10 includes an introducer 20 such as, for example, a needle cannula. In some embodiments, the introducer 20 has a diameter of less than or equal to 14 gauge (e.g., inner diameter of ≤1.6 mm). In some embodiments, the introducer may have a diameter greater than 14 gauge. The introducer has a tip 22, such as, for example, a sharp tip (e.g., lancet point grind, bias grind, razor edge, etc.) to allow easy insertion. An obturator 30 is disposed within the introducer 20 such that the obturator may slide (i.e., translate) within the introducer. The obturator 30 has a magnetic end 31. For example, the obturator 30 may be made up of a shaft 32 and a permanent magnet 34 at an end 33 of the shaft 32. The obturator is operable such that the magnetic end 31 is contained within the introducer 20 in a first position of the obturator, and the magnetic end 31 extends at least partially beyond the tip 22 of the introducer in a second position of the obturator.

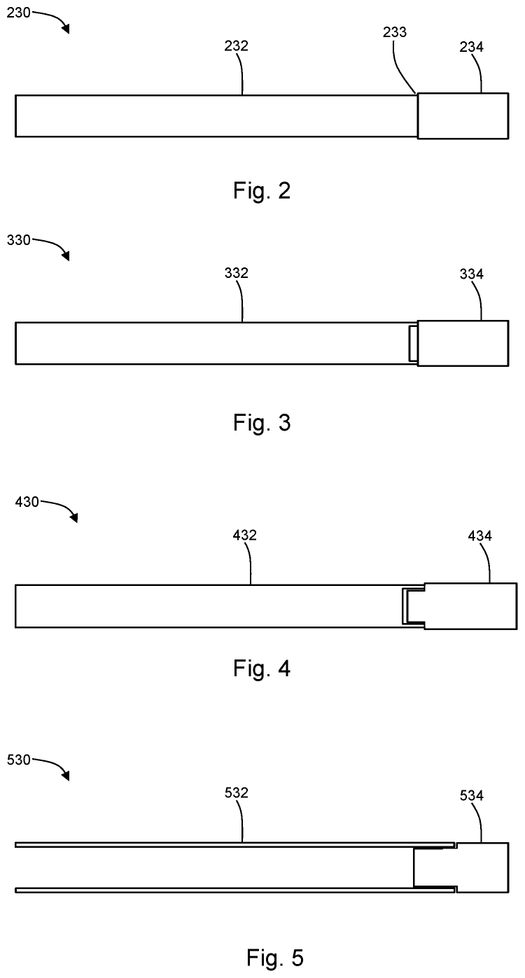

In some embodiments, where the obturator comprises a permanent magnet and a shaft, the permanent magnet may be adhered at an end of the shaft. For example, FIG. 2 depicts an embodiment of an obturator 230 where a permanent magnet 234 is abutted to an end 233 of a shaft 232. FIG. 3 depicts an embodiment of an obturator 330 where an end 333 of a shaft 332 is cupped and a permanent magnet 334 is adhered to the end of the shaft. Such an embodiment may advantageously allow room (i.e., volume) for additional adhesive within the cup of the shaft for affixing the permanent magnet to the end of the shaft. In FIG. 4, an obturator 430 has a permanent magnet 434 shaped to correspond with a cup-shaped end 433 of the shaft 432. In some embodiments, the permanent magnet is adhered to the shaft. In some embodiments, other techniques for affixing the magnet to the shaft may be used, such as, for example, a screw mechanism, an interference fit, soldering, brazing, welding, molding the magnet to an interlocking shape of the shaft (or molding the shaft to an interlocking shape of the magnet), or other techniques for affixation, or combinations of these and/or other techniques.

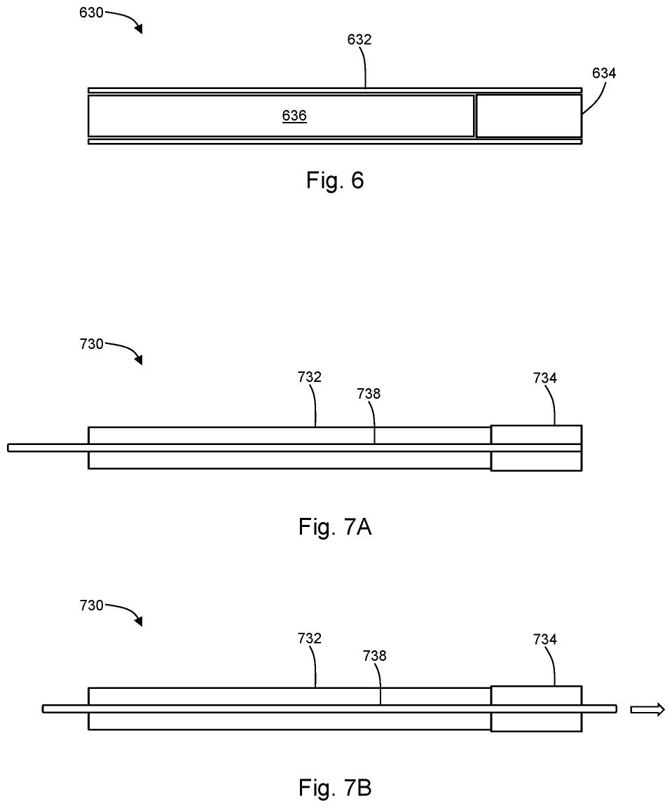

A shaft of an obturator may be made from one or more bio-compatible materials, such as, for example, surgical stainless steel, titanium, nitinol, polycarbonate, an acetyl-copolymer, polyether ether ketone (PEEK), etc. and/or other materials. In some embodiments, the shaft is made from combinations of these and/or other materials. In some embodiments, the shaft 532 of an obturator 530 is hollow. The permanent magnet 534 is affixed to the end of the shaft 532 using a mechanical connection (e.g., screw threads, interference fit, etc.), adhesive, and/or other technique (see FIG. 5). In some embodiments, the obturator 630 has a hollow shaft 632 and a rod 636 is inserted within the shaft 632 (see FIG. 6). The permanent magnet 634 may be adhered or otherwise connected to the rod 636. In the embodiment shown in FIG. 6, the permanent magnet 634 is shown contained within the hollow shaft 632. However, in other embodiments, such as that depicted in FIG. 5, the permanent magnet 534 is only partially disposed within the hollow shaft 532.

With reference to FIGS. 7A and 7B, in some embodiments, the device further includes an ejector 738 arranged to move through the obturator 730. For example, in FIG. 7A, the ejector 738 is coaxially disposed through the obturator 730 (e.g., through the shaft 732 and the permanent magnet 734 of the obturator). The ejector 738 may be operable to extend beyond the magnetic end 731 of the obturator 730. For example, the ejector 738 may have a first position wherein it is contained within the obturator (see FIG. 7A), and a second position wherein a portion of the ejector extends beyond the magnetic end 731 of the obturator 730. In other embodiments, the obturator may be disposed through the ejector (e.g., coaxially disposed through the ejector, etc.) Once a magnetic marker has been retrieved from a location within an individual, such an ejector may be used to detach the magnetic marker from the magnetic end of the obturator. In this way, the magnetic marker may be removed from a first location (e.g., using the magnet of the obturator) and placed in a second location in the individual (e.g., using the ejector to detach the magnetic marker from the obturator). The ejector may be made from one or more bio-compatible materials, such as, for example, surgical stainless steel, titanium, nitinol, polycarbonate, an acetyl-copolymer, PEEK, etc., and/or other materials. In some embodiments, the ejector is made from combinations of these and/or other materials.

The permanent magnet may comprise a bio-compatible magnetic material. The permanent magnet may be made from a magnetic material encapsulated by a bio-compatible material such as, for example, parylene. The permanent magnet may be made from or include neodymium. In some embodiments, the permanent magnet may include more than one magnet. For example, the permanent magnet may have a longitudinal (with reference to the obturator) stack of two or more magnets, such as, for example, disc magnets. In another example, the permanent magnet may include two or more magnets arranged along a direction transverse to a longitudinal axis of the obturator. In another example, a plurality of magnets may be arranged along both the longitudinal and transverse directions. In some embodiments, the permanent magnet is made up of one or more magnets and one or more pole pieces. For example, the permanent magnet may be an arrangement of two magnets with a central, ferrous pole piece.

In some embodiments, the introducer is sized to enter the skin minimally invasively to the target. Targeting a magnetic marker can be achieved though ultrasound (US), computed tomography (CT), magnetic resonance imaging (MRI), mammographic techniques, and/or other techniques.

In some embodiments, the magnetized component of the obturator has sufficient strength (i.e., of its magnetic field) to cause the magnetic marker to rotate (if needed-i.e., the magnetic poles were not arranged to attract) and align concentrically with the axis of the obturator and be captured by the obturator as it is magnetically attracted and attaches together. In such an orientation, the magnetic marker may be drawn into a lumen of the introducer for removal from the individual.

In some embodiments, the magnetized component serves two purposes:
Captures the marker, due to the magnetic field created by the magnetized component the implanted marker will attract to the obturator and will attach to it securing it so it can be removed.
Aligns the marker with the obturator, this allows for the captured magnetic marker to be removed from the patient through the cannula of the needle. If the poles do not align the marker may not fit down the lumen of the needle, making it impossible to remove from the patient.

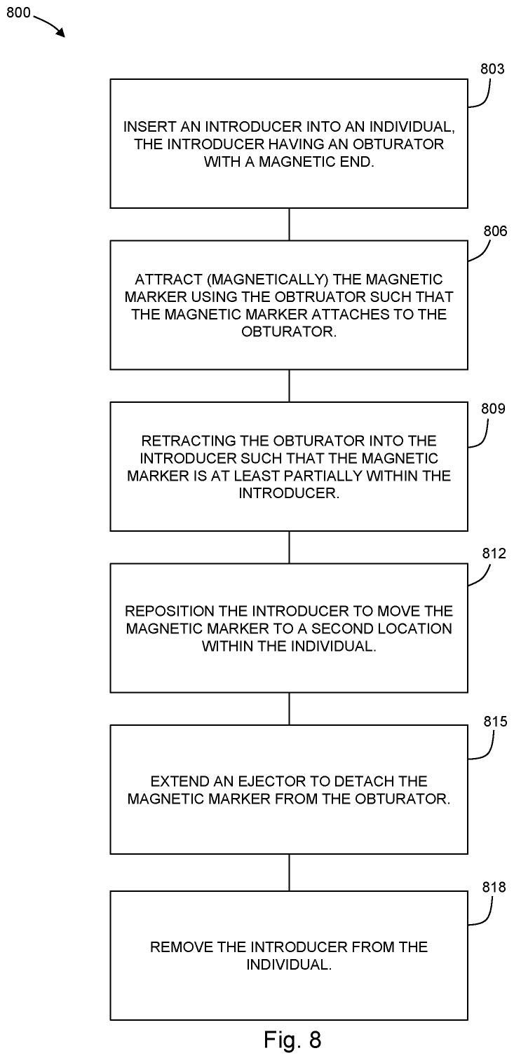

With reference to FIG. 8, in another aspect, the present disclosure may be embodied as a method 800 for removing a magnetic marker from a location (e.g., a first location) in an individual. The method 800 includes inserting 803 an introducer into an individual. The introducer has an obturator with a magnetic end. The method may use any of the device embodiments discloses herein. For example, the introducer may be a needle canula in which the obturator is disposed. The method 800 includes magnetically attracting 806 the magnetic marker using the obturator such that the magnetic marker attaches to the obturator. For example, the introducer may be moved such that a tip of the introducer is proximate to the magnetic marker. In some embodiments, the obturator may be moved through the introducer such that the magnetic tip is attracts the magnetic marker. As mentioned above, in some embodiments, the magnetic field of the magnetic tip of the obturator is sufficiently strong such that the magnetic marker is moved (rotated and/or translated) to attach to the obturator. The obturator may be retracted 809 into the introducer until the magnetic marker is at least partially within the introducer (e.g., at least partially surrounded by the introducer).

In some embodiments, the method 800 includes repositioning 812 the introducer to move the magnetic marker to a second location within the individual. An ejector may be extended 815 to detach the magnetic marker from the obturator. In this way, the magnetic marker may remain in the second location. The method may include removing 818 the introducer from the individual. The step of removing the introducer may include, for example, retracting the obturator into the introducer.

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof.

1. A device for retrieving a magnetic marker implanted in an individual, comprising:
an introducer having a tip; and
an obturator disposed within the introducer, the obturator having a magnetic end, wherein the obturator is operable such that the magnetic end is enclosed within the introducer in a first position of the obturator and the magnetic end extends at least partially beyond the tip of the introducer in a second position of the obturator.

2. The device of claim 1, wherein the obturator comprises a shaft and a permanent magnet affixed to the shaft at the magnetic end of the obturator.

3. The device of claim 2, wherein the permanent magnet is adhered at an end of the shaft.

4. (canceled)

5. (canceled)

6. (canceled)

7. The device of claim 2, wherein a portion of the permanent magnet is shaped to correspond with a cup-shaped end of the shaft.

8. The device of claim 2, wherein the shaft is hollow and the permanent magnet is at least partially disposed within an end of the hollow shaft.

9. (canceled)

10. The device of claim 2, wherein the permanent magnet comprises neodymium.

11. The device of claim 2, wherein the permanent magnet comprises a bio-compatible material.

12. The device of claim 11, wherein the permanent magnet comprises an enclosure comprising the bio-compatible material.

13. The device of claim 11, wherein the bio-compatible material is surgical stainless steel, titanium, nitinol, polycarbonate, an acetyl-copolymer, PEEK, or combinations thereof.

14. (canceled)

15. The device of claim 2, wherein the permanent magnet is mechanically affixed at an end of the shaft using screw threads, a dovetail fitting, or an interference fit.

16. The device of claim 1, further comprising an ejector arranged through the obturator and operable to extend beyond the magnetic end of the obturator.

17. The device of claim 16, wherein the ejector is coaxial with the obturator.

18. The device of claim 16, wherein the ejector comprises a bio-compatible material.

19. The device of claim 18, wherein the bio-compatible material is surgical stainless steel, titanium, nitinol, polycarbonate, an acetyl-copolymer, PEEK, or combinations thereof.

20. The device of claim 2, wherein the shaft comprises a bio-compatible material.

21. The device of claim 20, wherein the bio-compatible material is surgical stainless steel, titanium, nitinol, polycarbonate, an acetyl-copolymer, PEEK, or combinations of thereof.

22. The device of claim 2, wherein the permanent magnet comprises two or more magnets.

23. A method for removing a magnetic marker from a location within an individual, the method comprising:
inserting an introducer into an individual, the introducer having an obturator with a magnetic end;
magnetically attracting the magnetic marker using the obturator such that the magnetic marker attaches to the obturator; and
retracting the obturator into the introducer such that the magnetic marker is at least partially within the introducer.

24. The method of claim 23, wherein the magnetic marker is positioned in a first position within the individual, the method further comprising:
repositioning the introducer to move the magnetic marker to a second position within the individual; and
extending an ejector to detach the magnetic marker from the obturator.

25. The method of claim 22, further comprising removing the introducer from the individual.
